(19) United States
(12) Patent Application Publication
(10) Pub. No.: US 2011/0269205 A1
(43) Pub. Date: Nov. 3, 2011

(54) USE OF ENZYME CATALYSTS IN CO2 PCC PROCESSES

(75) Inventors: Meihong Su, Lyneham (AU); Victoria Haritos, O'Connor (AU)

(73) Assignee: COMMONWEALTH SCIENTIFIC AND INDUSTRIAL RESEARCH ORGANISATION, Campbell, Australian Capital Territory (AU)

(21) Appl. No.: 13/125,753

(22) PCT Filed: Oct. 23, 2009
(86) PCT No.: PCT/AU09/01396

§ 371 Date: Jul. 19, 2011

(30) Foreign Application Priority Data
Oct. 23, 2008 (AU) 2008905457

(51) Int. Cl. C12P 3/00

(52) U.S. Cl. 435/168

(57) ABSTRACT

A method for processing a stream enriched in CO₂ from a gas by the action of an absorbent in the stream includes desorbing CO₂ from the stream by application of heat to the stream to desorb the CO₂ and regenerate the absorbent in a reaction system that includes reconstitution of carbon dioxide and an alkanolamine from carbamate and ammonium ion solution. The energy requirement is materially reduced by the presence of a biocatalyst. In another aspect, a gas stream is contacted with a sorbent system to effect absorption of CO₂ from the gas stream, the sorbent and absorbed CO₂ are separated from the gas stream to form a CO₂-rich stream, and the sorbent system contains a primary or secondary alkanolamine and a catalyst selected to modify the reaction kinetics of the absorption process so as to materially increase the proportion of bicarbonate in the CO₂-rich stream relative to carbamate.

### FIELD OF THE INVENTION

This invention relates generally to the use of enzyme catalysts in the recovery of carbon dioxide from gas streams. The invention has particular application to CO₂ recovery from flue gases generated by coal- and gas-fired power plants or from process gases in a wide variety of industrial processes including steel plants, smelters, cement kilns and calciners. The ter m “process gases” refer to gas streams fed to or from a process, and embraces, e.g. syngas feed to an industrial furnace, and blast furnace gas in a steel plant.

### BACKGROUND OF THE INVENTION

There is rapidly growing pressure for stationary sources of CO₂ emissions such as power stations, to make step reductions in greenhouse gas (GHG) emissions through 1) capturing the CO₂ formed from the process, and 2) storing the CO₂ by various geological means. Most involve injecting CO₂ in a supercritical or “liquefied” state into deep aquifers, coal seams and adjacent strata, or at depth in the ocean, or converting the CO₂ into a solid mineral form.

In the case of power stations, as an example, there are at present three main approaches to CO₂ separation from new or existing power plants: 1) post combustion capture, 2) precombustion capture, and, 3) oxygen combustion with flue gas liquefaction. In this context, the present invention is primarily applicable to post combustion capture.

In post combustion (PCC) capture, the CO₂ in flue gases is preferentially separated from nitrogen and residual oxygen using a liquid solvent in an absorber. The CO₂ is then removed from the solvent in a process called desorption (or regeneration, and sometimes termed “stripping”), thus allowing the solvent to be reused. The desorbed CO₂ is liquefied by compression and cooling, with appropriate drying steps to prevent hydrate formation. The main disadvantage of this process is that the CO₂ partial pressure is relatively low (compared to the two alternative approaches mentioned above), which necessitates the use of CO₂ selective solvents. The regeneration of these solvents releases an essentially pure CO₂ stream, but this step is relatively energy intensive. Overall, this reduces the electrical power output by around 20%, due to the need to provide low temperature heat (approximately 65% of the total energy required) and work to drive the CO₂ liquefaction plant and other auxiliary equipment. Both heat and work are also required for dehydration of the liquefied product CO₂. The net effect is to reduce the thermal efficiency of the plant by around 9 percentage points.

Post combustion capture in this form is applicable to other stationary CO₂ sources, such as steel plants, cement kilns, calciners and smelters.

Amines in general, and alkanolamines in aqueous solution in particular, are a traditional class of liquid solvent for effecting the absorbent step in post combustion capture. Well known amines in this category are monoethanolamine (HOCH₂CH₂NH₂, known as MEA) and diethanolamine ((HOCH₂CH₂)₂NH, known as DEA), respectively examples of primary and secondary alkanolamines. With these solvents, a primary reaction with the carbon dioxide produces carbamate, which must then be hydrolysed to bicarbonate. However, with MEA and DEA, the carbamate compound is highly stable, due to the unrestricted rotation of the aliphatic carbon atom around the amino carbamate group. To overcome this disadvantage, a range of sterically hindered amines have been proposed: in this case the rotation of the alkyl group around the amino carbamate group is restricted, resulting in low stability of the carbamate compound and ready hydrolysis to bicarbonate.

Another proposal for enhancing the absorption stage of the post combustion process has been to employ biocatalysts to improve the reaction rate of the primary reactions. The usual catalyst proposed is carbonic anhydrase or its analogues. For example, international patent publication WO 2006/089423 proposes a formulation for the absorption of CO₂ that comprises water, any of a wide range of CO₂ absorption compounds, and a carbonic anhydrase as activator to enhance the absorption capacity of the CO₂ absorption compound. This compound is said to be preferably selected from the group consisting of amines, alkanolamines, dialkylether of polyalkylene glycols and mixtures thereof.

Other disclosures of interest in relation to biocatalysis of absorption reactions are to be found in US patent publications 2004/0219090 and US 2004/0259231, and in international patent publications WO 2004/028667 and WO 2004/056455.

It is an object of this invention to provide further enhancements of post combustion CO₂ capture processes through the use of biocatalysts and in preferred implementations by enzymatic catalysis.

It is not admitted that any of the information in this specification is common general knowledge, or that the person skilled in the art could reasonably be expected to have ascertained, understood, regarded it as relevant or combined it in any way at the priority date.

### SUMMARY OF THE INVENTION

In a first aspect, the invention redirects attention from the focus of the last several years on the absorption reaction and proposes applications of biocatalysts in the desorption or stripping stage of the post combustion capture (PCC) process.

In its first aspect, the invention provides a method of processing a stream enriched in CO₂ from a gas by the action of an absorbent in the stream, comprising
desorbing CO₂ from the stream by application of heat to the stream to desorb the CO₂ and regenerate the absorbent in a reaction system that includes reconstitution of carbon dioxide and an alkanolamine from carbamate and ammonium ion solution,
wherein the energy requirement for the aforementioned reconstitution is materially reduced by the present of a biocatalyst.

In certain embodiments of the invention, the aforementioned reconstitution may be represented by the following reaction sequence:

RNHCOO⁻+RNH₃⁺ [symbol] 2RNH₂+CO₂ (1)

where R is an alkanol group and RNH₂ is the reconstituted alkanolamine and is a primary or secondary alkanolamine.

Preferably the recovered CO₂ is separated and further treated, for example by being liquefied by compression and cooling.

Typically, the method of the first aspect is part of an overall cyclic post-combustion capture process that includes the earlier steps of cooling a stream of flue gases to a temperature suitable for efficient absorption of CO₂, contacting the stream of flue gases with a predetermined sorbent system to effect absorption of CO₂ from the stream of flue gases, separating the sorbent and absorbed CO₂ from the stream of flue gases to form a CO₂-rich stream, and effecting said desorbing step on the CO₂-rich stream.

For the first aspect of the invention, the biocatalyst may be an enzyme. A suitable enzyme may be selected from the group consisting of the hydrolase, lyase and ligase classes, although it is thought that, due, to their activity levels, one or more selected hydrolases may be preferred.

In accordance with an advantageous implementation of the first aspect of the invention, the biocatalyst is selected for its activity in cleaving urethane bonds to effect release of CO₂ and an amine. An insight of the present invention that has given rise to this implementation is the realisation that enzymes reported to be useful for the biodegradation of polyurethane would be applicable to the desorption of CO₂ from carbamate solutions because the -0-(CO)—N— functional group is common to urethane and to MEA carbamate solutions and it is the cleavage of this functional group that is catalysed by a biocatalyst in the biodegradation of polyurethane.

The thus selected biocatalyst may be a urethanase enzyme (EC 3.5.1.75). A suitable urethanase enzyme may be prepared from inter alia, Bacillus licheniformis, Rhodococcus equi and Citrobacter freundii. Urethanase enzymes have also been extracted from Lactobacillus casei and Exophiala jeanselmei.

The abbreviations “EC” and “EC numbers” herein, and accompanying notations, are references to the enzyme classification as established by the nomenclature committee of the International Union of Biochemistry and Molecular Biology (NC-IUBMD).

Also of particular interest may be the aliphatic amidohydroases and urethane amidohydrolase.

Other enzymes that may also be of interest as the biocatalyst present in a method according to the first aspect of the invention include the following:

Hydrolase class: members of the amidohydroases group with EC numbers 3.5.1.X, in particular 3.5.1.3 omega-amidases, 3.5.1.4 aliphatic amidases, 3.5.1.5 urease, 3.5.1.6 β-ureidopropionase, 3.5.1.53 N-carbamoylputrescine amidohydrolase, 3.5.1.54 urea-1-carboxylate amidohydrolase, 3.5.1.59 N-carbamoylsarcosine amidohydrolase (and related enzymes such as N-carbamyl-amino acid amidohydrolase) and 3.5.1.75 urethane amidohydrolase. Members of the esterase group with EC numbers 3.1.1.X including 3.1.1.1 carboxylesterase, 3.1.1.3 triacylalycerol lipase and 3.1.1.34 lipoprotein lipase. Also the peptide hydrolase group 3.4.X.X including 3.4.21.X serine endopeptidases and 3.4.24.X metalloendopeptidases.

Lyase class: members of the carboxy lyases (carbon-carbon lyases) especially all decarboxylases of EC numbers 4.1.1.1 through to 4.1.1.86. In particular EC 4.1.1.86 2,4-diaminobutanoate carboxy lyase. The decarboxylation enzymes such as those from the lyase class 4.1.1.X can be used in combination with carbonic anhydrase to speed up turnover of this class of enzyme as has been noted (Botre, F. Mazzei F (1999) Bioelectrochemistry and Bioenergetics 48: 463-467). Other carbon-nitrogen lyases are also enzymes with these activities especially EC 4.2.1.104 cyanate hydratase (cyanase) and 4.3.2.3 ureidoglycolate urea lyase.

Ligase class: members of the EC 6.3.X.X class are involved in C—N bond formation but act reversibly. Of particular note is EC 6.3.4.6 urea carboxylase that catalyses the reversible carboxylation of urea.

In a second aspect, the invention is directed to a process for recovering carbon dioxide from a gas stream, comprising:
contacting the gas stream with a sorbent system to effect absorption of CO₂ from the gas stream, and separating the sorbent and absorbed CO₂ from the gas stream to form a CO₂-rich stream;
wherein the sorbent system contains a primary or secondary alkanolamine and a catalyst, preferably a biocatalyst, selected to modify the reaction kinetics of the absorption process so as to materially increase the proportion of bicarbonate in the CO₂-rich stream relative to carbamate.

By increasing the proportion of bicarbonate relative to carbamate, the energy cost of a downstream desorption step, in which the CO₂ is separated from the CO₂-rich stream and the absorbent is regenerated, can be materially and advantageously reduced.

Preferably, the method in its second aspect includes the further step of desorbing CO₂ from the CO₂-rich absorbent stream by application of heat to the absorbent stream to desorb the CO₂ and regenerate the sorbent system. The separated CO₂ is preferably further treated, for example by being liquefied by compression and cooling.

Where the sorbent system contains a primary or secondary alkanolamine, the conventional principal reaction, i.e. in the absence of the selected biocatalyst of the invention, brings carbon dioxide into a solution as a carbamate according to the following reaction:

2RNH₂+CO₂ [symbol] RNHCOO⁻+RNH₃⁺ (2)

where R is an alkanol group. Enzymatic catalysis is thought to favour the following reaction system:

RNH₂+CO₂+H₂O [symbol] RNH₃+HCO₃⁻ (3)

By appropriate selection of the biocatalyst, the strong carbamate reaction can be relatively diminished in favour of the direct hydrolysis bicarbonate reaction.

A carbonic anhydrase is a suitable biocatalyst for the practice of the second aspect of the invention.

As used herein, except where the context requires otherwise the term “comprise” and variations of the term, such as “comprising”, “comprises” and “comprised”, are not intended to exclude other additives, components, integers or steps.

### EXAMPLE 1

### Preparation and Activity of a Suitable Urethanase

For urethanase production, a single clony of Bacillus licheniformis (ATCC#: 14580) was grown in 50 ml nutrient broth overnight at 37° C. with shaking at 200 rpm. The whole culture was then inoculated into fresh 500 ml nutrient broth and incubated at 37° C. with shaking at 160 rpm for another 12 hours. The cells were collected by centrifugation at 5000×g for 15 min at 4° C. The cells were resuspended in 20 mM Tris-Cl buffer (pH 7.5) and disrupted with the French pressure cell press. The cell-free extract was collected by centrifugation at 6000×g for 20 min at 4° C. The cell free extract (W1) was fractionated by precipitation with 0-20 (F1), 20-40 (F2), 40-60 (F3), 60-80 (F4) and 80-100% (F5) saturated ammonium sulphate. Each fraction was dialyzed against 20 mM Tris-Cl buffer, pH 7.5 to get rid of ammonium sulphate before test the enzyme activity by ammonia ion selective electrode.

Urethanase activity was first assessed by ammonia ion selective microelectrode (MI-740 Dip-type NH3 electrode, Microelectrodes, Inc., Bedford, N.H., USA) based on the amount of ammonia releasing from respective substrates of urethane, acetamide and butyl carbamate. In a typical experiment, 50 μL of cell-free extract was added to 250 μL of 100 mM urethane and acetamide, 4 mM butyl carbamate solutions. The reaction solutions were prepared in a 96 well plate and the reactions were carried out at 25° C. in 20 mM Tris-Cl buffer, pH 7.5. Measurements were taken after the reading from the electrode had stabilized. The results showed that the cell free extract and the F4 fraction (60-80% ammonium sulphate precipitation) had good urethanase activity with acetamide and butyl carbamate (table 1).

The F4 fractions that showed good initial urethanase activity were further purified by an ion exchange chromatography and eluted with a linear gradient of sodium chloride (0-0.5M). Urethanase activity of each fraction was assessed by ammonia ion selective microelectrode based on the amount of ammonia released from the substrates, mentioned in the previous step. The fractions exhibiting urethanase activity were pooled and concentrated by ultrafiltration.

Further purification was achieved by running size exclusion chromatography. The concentrated enzyme solution from the preceding ion exchange purification was directly applied to a sepharose column and eluted with 20 mM Tris buffer, pH 7.5. The enzyme activity was tested by microelectrode based on the amount of ammonia released from the substrates, mentioned earlier. The active fractions were pooled and concentrated by ultrafiltration.

SDS-PAGE was then run to test the purification of urethanase. Hydroxyapatite chromatography and isoelectric chromatofocusing can be applied if further purification is required.

TABLE 1
Enzyme activity of cell free extract (W1), 0-20 (F1), 20-40 (F2),
40-60 (F3), 60-80 (F4) and 80-100% (F5) fractions against three substrates

| | | | | | |
|---|---|---|---|---|---|
| Urethane | Acetamide | Butyl | | | |
| (−)* | (−) | carbamate | | | |
| | | (−) | | | |
| Cell free (−) | F1 (−) | F2 (−) | F3 (−) | F4 (−) | F5 (−) |
| Urethane and | Urethane | Urethane | Urethane | Urethane | Urethane |
| cell free (+) | and F1 (−) | and F2 (−) | and F3 (−) | and F4 (+) | and F5 (−) |
| Acetamide | Acetamide | Acetamide | Acetamide | Acetamide | Acetamide |
| and cell free | and F1 (−) | and F2 (−) | and F3 (−) | and F4 | and F5 (−) |
| (++) | | | | (++) | |
| Butyl | Butyl | Butyl | Butyl | Butyl | Butyl |
| carbamate | carbamate | carbamate | carbamate | carbamate | carbamate |
| and cell free | and F1 (−) | and F2 (−) | and F3 (+) | and F4 | and F5 (+) |
| (+++) | | | | (+++) | |

*
+++: high activity
++: middle activity
+: some activity
−: no activity

### EXAMPLE 2

### Bicarbonate Production in Mea Solution with and without a Biocatalyst

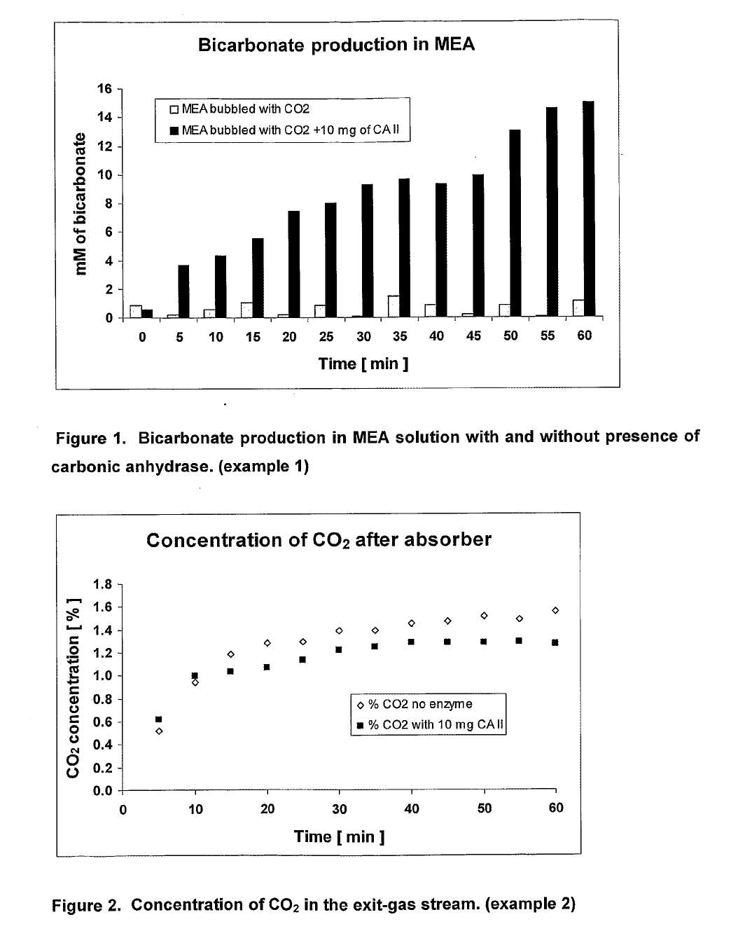

50 mL of freshly prepared 0.5 M monoethanolamine (MEA) solution in D₂O was poured into a 125 mL Dreschel bottle and 80 μL of the solution was taken as a zero time sample (control). 100 mL min⁻¹ of 2% CO₂ in 98% N₂ was bubbled into the stirred solution through the sintered glass outlet via a silicon tube in the bottle head. A subsample of the solution (80 μL) was taken every 5 minutes and the IR absorbance was immediately measured between 1300 to 1700 cm⁻¹. The bicarbonate peak at 1630 cm⁻¹ was used as it was free of background noise. The area under the peak at 1630 cm⁻¹ was calculated for each sample to provide the concentration of bicarbonate over a period of 60 minutes. The experiment was repeated under the same conditions in the presence of 10 mg of carbonic anhydrase (CA II). The results are shown in FIG. 1.

The concentration of bicarbonate produced in the MEA solution without enzyme was less than 2 mM and then did not increase during the 60 minutes period of bubbling. In the solution with CA II, initial production of bicarbonate was low due to formation of MEA carbamate, which is the faster reaction. The kinetics of CO₂ absorption were obtained from measurements of the CO₂ concentration in the exit-gas stream from the Dreschel bottle shown in FIG. 2. After the initial period of MEA-carbamate formation, the concentration of bicarbonate increased linearly, reaching 14.9 mM at the end of the 60 minutes (FIG. 1).

The reduced concentration of CO₂ in the exit-gas stream in the presence of CA II as shown in FIG. 2 indicates that more CO₂ is absorbed in the MEA solution in the presence of the enzyme compared to that without enzyme. The increased absorption of CO₂ results in higher concentration of bicarbonate produced in the MEA solution in the presence of CA II compared to that without enzyme. It is therefore concluded that the presence of enzyme has altered the final ratio between produced carbamate and bicarbonate. Higher production of bicarbonate in MEA solution is favorable for industrial application due to lower energy demand for bicarbonate regeneration, resulting in lower regeneration costs.

1. A method of processing a stream enriched in CO₂ from a gas by the action of an absorbent in the stream, comprising:
desorbing CO₂ from the stream by application of heat to the stream to desorb the CO₂ and regenerate the absorbent in a reaction system that includes reconstitution of carbon dioxide and an alkanolamine from carbamate and ammonium ion solution,
wherein the energy requirement for the aforementioned reconstitution is materially reduced by the presence of a biocatalyst.

2. The method according to claim 1, wherein the reconstitution is represented by the following reaction sequence:
RNHCOO⁻+RNH₃⁺ [symbol] 2RNH₂+CO₂
where R is an alkanol group and RNH₂ is the reconstituted alkanolamine and is a primary or secondary alkanolamine.

3. The method according to claim 1, wherein the recovered CO₂ is separated and further treated, by being liquefied by compression and cooling.

4. The method according to claim 2, wherein the method is part of an overall cyclic post-combustion capture process that includes the earlier steps of cooling a stream of flue gases to a temperature suitable for efficient absorption of CO₂, contacting the stream of flue gases with a predetermined sorbent system to effect absorption of CO₂ from the stream of flue gases, separating the sorbent and absorbed CO₂ from the stream of flue gases to form a CO₂-rich stream, and effecting said desorbing step on the CO₂-rich stream.

5. The method according to claim 1, wherein the biocatalyst is an enzyme.

6. The method according to claim 5, wherein the enzyme is selected from the group consisting of the hydrolase, lyase and ligase classes.

7. The method according to claim 6, wherein the hydrolase class includes members of the amidohydroases group with EC numbers 3.5.1.X, including 3.5.1.3 omega-amidases, 3.5.1.4 aliphatic amidases, 3.5.1.5 urease, 3.5.1.6 β-ureidopropionase, 3.5.1.53 N-carbamoylputrescine amidohydrolase, 3.5.1.54 urea-1-carboxylate amidohydrolase, 3.5.1.59 N-carbamoylsarcosine amidohydrolase (and related enzymes such as N-carbamyl-amino acid amidohydrolase) and 3.5.1.75 urethane amidohydrolase, members of the esterase group with EC numbers 3.1.1.X including 3.1.1.1 carboxylesterase, 3.1.1.3 triacylalycerol lipase and 3.1.1.34 lipoprotein lipase, and members of the peptide hydrolase group 3.4.X.X including 3.4.21.X serine endopeptidases and 3.4.24.X metalloendopeptidases.

8. The method according to claim 6, wherein the lyase class includes members of the carboxy lyases (carbon-carbon lyases) including all decarboxylases of EC numbers 4.1.1.1 through to 4.1.1.86, EC 4.1.1.86 2,4-diaminobutanoate carboxy lyase, and carbon-nitrogen lyases including EC 4.2.1.104 cyanate hydratase (cyanase) and 4.3.2.3 ureidoglycolate urea lyase.

9. The method according to claim 6, wherein the ligase class includes members of the EC 6.3.X.X class including EC 6.3.4.6 urea carboxylase.

10. The method according to claim 1 wherein the biocatalyst is selected for its activity in cleaving urethane bonds to effect release of CO₂ and an amine.

11. The method according to claim 10 wherein the biocatalyst is a urethanase enzyme.

12. The method according to claim 11 wherein the urethanase enzyme has been prepared from one of Bacillus licheniformis, Rhodococcus equi, Citrobacter freundii, Lactobacillus casei and Exophiala jeanselmei.

13. The method according to claim 1 wherein the biocatalyst is selected from aliphatic amidohydroases and urethane amidohydrolases.

14. The method according to claim 6, wherein the enzyme is one of cyanate hydratase (cyanase), EC number: 4.2.1.104 and N-carbamoylputrescine amidohydrolase, EC number: 3.5.1.53.

15. The method according to claim 6, wherein the enzyme is one of beta-ureidopropionase, EC number: 3.5.1.6 and N-carbamoylsarcosine amidohydrolase, EC number: 3.5.1.59.

16. A process for recovering carbon dioxide from a gas stream, comprising:
contacting the gas stream with a sorbent system to effect absorption of CO₂ from the gas stream, and separating the sorbent and absorbed CO₂ from the gas stream to form a CO₂-rich stream;
wherein the sorbent system contains a primary or secondary alkanolamine and a catalyst selected to modify the reaction kinetics of the absorption process so as to materially increase the proportion of bicarbonate in the CO₂-rich stream relative to carbamate.

17. The process according to claim 16, wherein the catalyst is a biocatalyst.

18. The process according to claim 16, further including the step of desorbing CO₂ from the CO₂-rich absorbent stream by application of heat to the absorbent stream to desorb the CO₂ and regenerate the sorbent system.

19. The process according to claim 18, wherein the separated CO₂ is further treated by being liquefied by compression and cooling.

20. The process according to claim 16, wherein the catalyst is a carbonic anhydrase.

21. The method according to claim 4, wherein the biocatalyst is an enzyme.

22. The method according to claim 21, wherein the enzyme is selected from the group consisting of the hydrolase, lyase and ligase classes.

23. The method according to claim 2 wherein the biocatalyst is selected for its activity in cleaving urethane bonds to effect release of CO₂ and an amine.

24. The method according to claim 23, wherein the biocatalyst is an enzyme.

25. The method according to claim 23 wherein the biocatalyst is a urethanase enzyme.

26. The method according to claim 25 wherein the urethanase enzyme has been prepared from one of Bacillus licheniformis, Rhodococcus equi, Citrobacter freundii, Lactobacillus casei and Exophiala jeanselmei.

27. The method according to claim 23, wherein the method is part of an overall cyclic post-combustion capture process that includes the earlier steps of cooling a stream of flue gases to a temperature suitable for efficient absorption of CO₂, contacting the stream of flue gases with a predetermined sorbent system to effect absorption of CO₂ from the stream of flue gases, separating the sorbent and absorbed CO₂ from the stream of flue gases to form a CO₂-rich stream, and effecting said desorbing step on the CO₂-rich stream.

28. The method according to claim 2 wherein the biocatalyst is selected from aliphatic amidohydroases and urethane amidohydrolases.

29. The method according to claim 4 wherein the biocatalyst is selected from aliphatic amidohydroases and urethane amidohydrolases.

30. The process according to claim 17, further including the step of desorbing CO₂ from the CO₂-rich absorbent stream by application of heat to the absorbent stream to desorb the CO₂ and regenerate the sorbent system.

31. The process according to claim 20, further including the step of desorbing CO₂ from the CO₂-rich absorbent stream by application of heat to the absorbent stream to desorb the CO₂ and regenerate the sorbent system.
